(19) United States
(12) Patent Application Publication
(10) Pub. No.: US 2003/0157200 A1
(43) Pub. Date: Aug. 21, 2003

(54) Use of oligosaccharides to stimulate beta-endorphin production

(76) Inventors: Frederic Bonte, Orleans (FR); Marc Dumas, Orleans (FR); Stephane Lhermite, Semoy (FR); Alex Saunois, Orleans (FR)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 10/332,136

(22) PCT Filed: Jul. 5, 2001
(86) PCT No.: PCT/FR01/02154

(30) Foreign Application Priority Data
Jul. 7, 2000 (FR) 00/08879

(51) Int. Cl. A61K 35/78; A61K 31/715

(52) U.S. Cl. 424/725; 514/61; 424/761

(57) ABSTRACT

The invention concerns the use of at least an oligosaccharide comprising 2 to 6 sugars and containing at least 2 galactose units, preferably 2 vicinal end-of-chain galactose motifs, or a plant extract containing same, as a cosmetic or dermatological agent, in particular for stimulating the beta-endorphin production in the skin. The invention enables to provide care for sensitive skins, in particular to fight against skin sensitivity, uncomfortable reactions, to provide sensations of well-being, and to produce a local analgesic action.

The present invention relates essentially to the use of oligosaccharides containing at least two galactose units, or a plant extract containing them, as cosmetic or dermatological agents.

The present invention relates essentially to the use of oligosaccharides containing at least two galactose units, or a plant extract containing them, as cosmetic or dermatological agents, and to a method of cosmetic care in which they are applied. More particularly, the invention relates to the use of oligosaccharides comprising from 2 to 6 sugars and containing at least two galactose units, preferably two vicinal galactose units and more preferably two vicinal galactose units at the end of the chain, or of a plant extract containing them, as cosmetic agents or for the manufacture of a pharmaceutical composition, particularly a dermatological composition, notably for stimulating the production of beta-endorphin in the skin and preferably for stimulating the production of beta-endorphin by the keratinocytes of the skin, and to a method of cosmetic care or a method of therapeutic treatment in which they are applied.

The following may be mentioned in particular among the oligosaccharides containing at least two vicinal galactose units: D-stachyose, which is more commonly called stachyose or called O-α-D-galactopyranosyl-[1→6]-O-α-D-galactopyranosyl-[1→6]-β-D-fructofuranosyl-α-D-glucopyranoside, of the empirical chemical formula C₂₄H₄₂O₂₁, and is commercially available, or ciceritol, or O-α-D-galactopyranosyl-[1→6]-O-α-D-galactopyranosyl-[1→2]-4-O-methyl-D-chiroinositol, of the empirical chemical formula C₁₉H₃₄O₁₆.

These oligosaccharides can be isolated from plants, particularly from a plant of the genus Tephrosia and in particular from the species Tephrosia purpurea.

These oligosaccharides can also be isolated from a plant of the soya, chick pea, lupin or lentil type.

The use of extracts of Tephrosia, in particular Tephrosia purpurea, has already been described in document FR-2 708 198 B for the preparation of a cosmetic or pharmaceutical composition, notably a dermatological composition, and a method of cosmetic treatment in which it is applied on the basis of the unexpected discovery that such an extract has a potent stimulating activity on the enzyme adenylate cyclase.

Within the context of the present invention, it has now been discovered, totally unexpectedly, that certain oligosaccharides comprising from 2 to 6 sugars and having at least 2 galactose units, preferably 2 vicinal galactose units and more preferably two vicinal galactoses at the end of the chain, or a plant extract containing them, notably an extract of the plant Tephrosia and in particular Tephrosia purpurea, are capable of stimulating the production of β-endorphin in the skin and preferably of stimulating the production of β-endorphin by the keratinocytes of the skin.

One main object of the present invention is thus to solve the new technical problem consisting in the provision of a solution for obtaining novel cosmetic or dermatological agents, or novel cosmetic or dermatological compositions, capable of stimulating the production of β-endorphin in the skin and notably of stimulating the production of β-endorphin by the keratinocytes of the skin.

Another main object of the present invention is to solve the new technical problem consisting in the provision of a solution for obtaining novel cosmetic or dermatological agents, or pharmaceutical compositions, notably dermatological compositions, capable of caring for sensitive skin, combating skin sensitivity and uncomfortable reactions, providing a sensation of well-being and having a soothing, anti-irritant, antipruritic or local analgesic effect.

These technical problems are solved by the present invention for the first time in a particularly simple, reliable and reproducible cosmetic or pharmaceutical manner that can be used on the industrial scale.

Thus, according to a first feature, the present invention covers the use of at least one oligosaccharide comprising from 2 to 6 sugars and containing at least 2 galactose units, preferably 2 vicinal galactose units and more preferably two vicinal galactose units at the end of the chain, or of a plant extract containing it, as a cosmetic or dermatological agent for stimulating the production of beta-endorphin in the skin.

In another advantageous embodiment, the use is characterized in that the above-mentioned oligosaccharide is stachyose.

In another advantageous embodiment of the invention, the above-mentioned oligosaccharide is ciceritol.

In one advantageous embodiment within the context of the invention, it is possible to use an extract of plant seeds containing the oligosaccharide defined above, the plant preferably being selected from the group comprising Tephrosia, soya, chick pea, lupin and lentil. More preferably, the plant extract comes from seeds of the species Tephrosia purpurea.

In one advantageous embodiment, the seed extract is an aqueous-alcoholic extract using a linear, branched or cyclic C₁-C₆ alcohol. A particularly preferred alcohol is methanol, ethanol or butanol. The relative water/alcohol proportions can vary within wide limits. The currently preferred mixture is about ⅔ of alcohol to ⅓ of water in relative proportions by weight. The ratio of weight of solvent/weight of starting materials that can be used for this extraction is from about 5/1 to about 50/1 or more, and is preferably in the order of about 12/1.

The extraction can be carried out at room temperature or with any moderate heating, particularly at an extraction temperature of between 20° C. and 70° C. and preferably at about 45° C. This gives a concentrated aqueous product which can be resolubilized e.g. after addition of the same alcohol or a different alcohol, especially propylene glycol or ethanol, or a large volume of water. It is currently advantageous to carry out a triple formulation of the product in an alcohol/water mixture in the order of 30/70 by weight, the alcohol preferably being propylene glycol. It is also possible to add a surfactant such as Phénonip®.

The extract obtained is composed essentially of oligosaccharides and more precisely of fructose, sucrose, raffinose, stachyose and ciceritol.

In yet another advantageous embodiment of the invention, the above-mentioned oligosaccharide, or a plant extract containing it, is combined with another cosmetically or dermatologically acceptable active substance preferably selected from the group consisting of vitamin A and its esters, particularly vitamin A palmitate; an alpha-hydroxy acid, particularly salicylic acid and its derivatives or lactic, glycolic or malic acid; an inhibitor of the enzyme PLA2, such as an extract of the plant Phellodendron amurense or the plant Azadirachta indica; a substance with anti-inflammatory activity, such as 18B-glycyrrhetinic acid, an extract of the plant Glycyrrhiza glabra; a substance with immunomodulating activity, such as a glycan; a surfactant, particularly of the laurylsulfate family; an alkaloid substance, preferably a bisbenzylisoquinoline and particularly oxyacanthine or cepharanthine; a PAF inhibitor, particularly a Gingko biloba extract; and an inhibitor of PGE2 enzymes.

In yet another advantageous embodiment of the invention, the above-mentioned oligosaccharide, or a plant extract containing it, is applied to the skin in order to care for sensitive skin, notably to reduce or eliminate uncomfortable reactions, provide a sensation of well-being or exert a local analgesic action.

According to a second feature, the present invention also covers a method of cosmetic care, characterized in that it comprises the application, to the areas of skin in question, of a cosmetically effective amount of at least one oligosaccharide comprising from 2 to 6 sugars and containing at least 2 galactose units, preferably 2 vicinal galactose units and more preferably two vicinal galactose units at the end of the chain, or of a plant extract containing it, optionally in a cosmetically acceptable excipient.

According to a third feature, the present invention also covers a method of therapeutic treatment, in particular for soothing pain and combating itching, characterized in that it comprises the administration, to the skin areas of the person in question, of a therapeutically effective amount of at least one oligosaccharide comprising from 2 to 6 sugars and containing at least two galactose units, preferably two vicinal galactose units and more preferably two vicinal galactose units at the end of the chain, or of a plant extract containing it, optionally in a pharmaceutically acceptable excipient, preferably for carrying out a therapeutic treatment involving stimulation of the production of β-endorphin in the skin.

In another advantageous embodiment, from 0.0001% to 10%, preferably 0.01 to 5%, of oligosaccharides or extracts containing them, expressed by dry weight based on the total weight of the composition, will be used in any one of the preceding features.

Other objects and advantages of the invention will become clearly apparent from the following explanatory description referring to various Examples of the invention, which are given simply by way of illustration and cannot in any way limit the scope of the invention. However, the Examples form an integral part of the present invention and any characteristic which might appear novel relative to any state of the art forms part of the invention in its generality and is claimed as such. In the description and the claims, unless indicated otherwise, the percentages are given by weight, the temperatures are in degrees Celsius and the pressure is atmospheric pressure.

### EXAMPLE 1

### Preparation of an Extract Rich in Oligosaccharides, Particularly Oligosaccharides having 2 Vicinal Galactose Units Located at the end of the Chain (Product I₁ of the Invention)

100 grams of commercially available Tephrosia purpurea seeds are ground and passed through a 1 mm screen. These ground seeds are introduced into a mixture of extraction solvent and water comprising about 0.84 kg of alcohol and about 0.36 kg of distilled water, i.e. an aqueous-alcoholic extraction mixture containing about 70% by weight of alcohol.

Within the context of this Example, the extraction alcohol is 96% v/v ethanol.

An extraction is performed with this extraction mixture for about 5 hours at about 45° C., with agitation.

The mixture is then cooled to room temperature, i.e. about 25° C. It is filtered under vacuum using a filter with a pore size of about 11 μm.

In the same reactor the mixture is concentrated under a vacuum pressure of between 160 and 60 mbar and at a vacuum concentration bath temperature of about 58° C. to give a concentrate of about 80 g.

After evaporation, the residue is redissolved, still in the same reactor, by the addition of an alcohol, in this case propylene glycol of cosmetic grade, in an amount of about 30 g, with vigorous agitation for about 20 min.

This product can be used as such or can be purified by the addition of 14 g of commercial active carbon (reference C x V, CECA, France) to the solution, which is agitated for 15 min at room temperature.

A conventional vacuum filtration is then carried out on a filter with a pore diameter of 5 μm.

The weight of the filtered solution containing the oligosaccharides is about 70 g. The filtration proceeds without problems. The solids content is adjusted to about 5% by the addition of a propylene glycol/water mixture of 30/70 by weight.

A further filtration is carried out on a filter with a pore diameter of 0.22 μm, after which it is optionally and advantageously possible to add a surfactant, such as Phénonip®, in an amount of 0.5% by weight. The resulting product is called product I₁ of the invention.

### EXAMPLE 2

The procedure is as described in Example 1 except that butanol is used as the extraction alcohol.

This gives a product of the invention called product I₂.

### EXAMPLE 3

The procedure is as described in Example 1 except that methanol is used as the extraction alcohol.

This gives a product of the invention called product I₃.

### EXAMPLE 4

### Preparation of Purified Ciceritol from Tephrosia purpurea Seeds

The procedure is as described in Example 1 as far as the vacuum concentration step to give about 80 g of concentrate in the reactor, the vacuum concentration bath temperature being about about 58° C. and the vacuum pressure being between 160 and 60 mbar.

14 g of commercial active carbon (reference C x V from CECA, France) are then added and the mixture is agitated for 15 min at room temperature.

A vacuum filtration is then carried out in conventional manner on a filter with a pore diameter of 5 μm.

A further filtration is then carried out on a filter with a pore diameter of 0.2 μm, after which the filtrate is evaporated to dryness.

The yield obtained is 7% by dry weight.

The product is then subjected to high performance preparative liquid chromatography in the following manner:

Experimental Conditions

A steel column with axial compression (ID=4 cm/length=30 cm) is used.

Stationary phase: Lichrospher 100 DIOL® 15 μm (Merck)

Packing of the column: 200 g of stationary phase are dispersed in acetonitrile.

Pressure/compression: 100 bar

Amount injected: 630 mg of dried extract diluted in 3 ml of water

$$\begin{matrix}Elution gradient \text{:} acetonitrile : 95 \\ water : 5\end{matrix} {⟶}^{72 min} \begin{matrix}acetonitrile : 50 \\ water : 50\end{matrix}$$

Elution rate: 100 ml.min⁻¹

Detection: SEDEX 55 photomultiplying light-scattering detector (Sedere, Alfortville, France) (PM 4 at 2.5 bar of air and 45° C.).

The purified product eluted is evaporated and then lyophilized to give a white powder with a decomposition point of 160° C. and an optical rotation [α]_{D}²⁵ of +159.01° in water at 0.93 g/ml.

The purification yield is about 7%, giving an overall yield of 0.49% based on the dry extract, the purity being greater than 90%. The purity is checked in a similar manner by high performance liquid chromatography on the same analytical column of the DIOL® type, this check revealing the presence of a single molecule of very high purity. A structural study by both NMR and mass spectrography provided confirmation that the compound obtained did indeed have the structure of ciceritol.

The product obtained in this way is used in the experiments of Example 5 below.

### EXAMPLE 5

### Demonstration of the Stimulating Action of Tephrosia purpurea Extracts, Stachyose and Ciceritol on the Synthesis of Beta-Endorphin by Normal Human Keratinocytes

It is described in the literature, notably in J. Invest. Dermatol. 1996, 106, 673-678; J. Clin. Invest. 1994, 93, p. 2258-2262, that human keratinocytes—cells which constitute one of the essential components of the epidermis—are capable of synthesizing and secreting certain neurohormones such as beta-endorphin. Beta-endorphin is a derivative of propiomelanocortin (POMC), which very probably has a role in immunomodulation phenomena and in the hair cycle, as described in the literature, notably in J. Invest. Dermatol. 1996, 106, 3-10; Biochim. Biophys. Acta 1997, 1336, p. 315-322.

The hypothesis has been put forward that the release of beta-endorphin brought about by the keratinocytes is sufficient to enter the serum and act remotely on the central nervous system and the circulating immune cells, as described in J. Invest. Dermatol. 1996, 106, 673-678.

POMC, a hormone originally discovered in the pituitary gland, exerts the functions of neuropeptide precursors. Neurohormones are released into the organism during stress situations or UV irradiation and have analgesic effects which can be important for the development of cosmetic products intended in particular for sensitive skin.

A specific role of β-endorphin compared with enkephalins has been demonstrated in the literature. For example, in Exp. Dermatol. 1997, 6, 222-229, Nissen J. B. et al. demonstrated that, in contrast to enkephalins, β-endorphin possessed no role in keratinocyte differentiation.

In the present experiment, the inventors demonstrated, totally unexpectedly, that oligosaccharides having at least 2 vicinal galactose sugars, preferably at the end of the chain, particularly the oligosaccharides present in Tephrosia purpurea extracts and specifically stachyose and ciceritol, had the capacity significantly to stimulate the synthesis of β-endorphin by normal human keratinocytes. The activity test is as follows:

Activity Test

Test Products

The test uses either aqueous-alcoholic Tephrosia purpurea extracts obtained by the process of Example 1, or stachyose available commercially (from Sigma, France), or ciceritol isolated from the aqueous-alcoholic Tephrosia purpurea extract I₁ of Example 1, as described in Example 4.

Cell Test

Norman human keratinocytes are cultivated to the point of confluence on a 24-well plate and then incubated in a culture medium for 24 hours in the presence of dibutiryl CAMP (2 mM), interleukin-1β (IL-1β) (500 pg/ml) and the test product, i.e. in this case either Tephrosia purpurea extracts according to Example 1, or stachyose, or ciceritol, at the doses indicated in Tables I, II and III respectively.

### 5.1. Experiment with Tephrosia Extracts

Each Tephrosia extract obtained by the process of Example 1 is provided accurately weighed and redissolved at a concentration of 50 mg/ml in an ethanol/water mixture (1/1).

Several extraction batches of Tephrosia purpurea seeds were prepared by the extraction procedure described in Example 1, the batches being called B1, B2 and B3 respectively. Two series of experiments were performed on batch B3.

The results obtained as indicated below are listed in Table I.

Antiproteases—aprotinin 5 μg/ml, leupeptin 1 μg/ml and PMSF 1 mM —are added to each well in order to limit the action of the proteases.

Each experimental point is duplicated.

After incubation for 24 h, the culture supernatants are recovered and frozen at −80° C.

Positive stimulation controls are carried out in parallel, the cells being treated for 24 h, as above, with either IL-1β 500 pg/ml or dibutyryl CAMP 2 mM.

The β-endorphin secreted is assayed by EIA (kit from Peninsula Laboratoiries) and expressed in pg/ml.

The results obtained are indicated in Table I below:

TABLE I

| | | Student t |
|---|---|---|
| Experimental conditions of | β-endorphin | test |
| keratinocyte treatment | secreted in pg/ml | value of p |
| 0.025% ethanol control | 36 ± 6 | |
| Tephrosia purpurea 1 μg/ml (B1) | 74 ± 8 | 0.0359 |
| Tephrosia purpurea 5 μg/ml (B1) | 98 ± 15 | 0.0323 |
| Tephrosia purpurea 25 μg/ml (B1) | 77 ± 11 | 0.0456 |
| Control | 3 ± 4 | |
| Positive control: IL-1β 500 pg/ml | 35 ± 5 | 0.0208 |
| Positive control: dibutyryl cAMP 2 mM | 87 ± 12 | 0.0115 |
| Control | 3 ± 4 | |
| Tephrosia purpurea 1 μg/ml (B2) | 26 ± 6 | 0.0442 |
| Tephrosia purpurea 5 μg/ml (B2) | 44 ± 7 | 0.0196 |
| Tephrosia purpurea 25 μg/ml (B2) | 26 ± 4 | 0.0324 |
| Positive control: IL-1β 500 pg/ml | 35 ± 5 | 0.0208 |
| Positive control: dibutyryl cAMP 2 mM | 87 ± 12 | 0.0115 |
| Control | 1 ± 1 | |
| Tephrosia purpurea 1 μg/ml (B3) | 3 ± 1 | 0.3081 |
| Tephrosia purpurea 5 μg/ml (B3) | 14 ± 2 | 0.0047 |
| Tephrosia purpurea 25 μg/ml (B3) | 14 ± 2 | 0.0047 |
| Positive control: IL-1β 500 pg/ml | 17 ± 3 | 0.0036 |
| Positive control: dibutyryl cAMP 2 mM | 10 ± 1 | 0.0065 |
| Control | 1 ± 3 | |
| Tephrosia purpurea 1 μg/ml (B3) | 21 ± 5 | 0.0280 |
| Tephrosia purpurea 5 μg/ml (B3) | 21 ± 4 | 0.0179 |
| Tephrosia purpurea 25 μg/ml (B3) | 20 ± 1 | 0.0121 |
| Positive control: IL-1β 500 pg/ml | 25 ± 4 | 0.0065 |
| Positive control: dibutyryl cAMP 2 mM | 22 ± 1 | 0.0067 |

### 5.2 Experiments with Stachyose and Ciceritol

The procedure followed is the same as in the experiments with Tephrosia purpurea extracts except that stachyose and ciceritol are used at the doses indicated in Table II, and the results obtained are indicated in Table II below.

TABLE II

| | β-endorphin | Student t |
|---|---|---|
| Experimental conditions of | secreted in | test |
| keratinocyte treatment | pg/ml | value of p |
| Control | 10 ± 3 | |
| Stachyose 20 ng/ml (Sigma ref. | 16 ± 1 | 0.0733 |
| S4001) | | |
| Stachyose 100 ng/ml (Sigma ref. | 22 ± 2 | 0.0086 |
| S4001) | | |
| Stachyose 500 ng/ml (Sigma ref. | 30 ± 13 | 0.0289 |
| S4001) | | |
| Positive control: IL-1β 500 pg/ml | 25 ± 4 | 0.0065 |
| Positive control: dibutyryl cAMP 2 mM | 22 ± 1 | 0.0067 |
| Control | 1 ± 2 | |
| Ciceritol 20 ng/ml | 8 ± 3 | 0.0380 |
| Ciceritol 100 ng/ml | 8 ± 4 | 0.0765 |
| Ciceritol 500 ng/ml | 9 ± 5 | 0.0863 |
| Positive control: IL-1β 500 pg/ml | 17 ± 3 | 0.0036 |
| Positive control: dibutyryl cAMP 2 mM | 10 ± 1 | 0.0065 |
| Control | 3 ± 3 | |
| Ciceritol 64 ng/ml | 44 ± 7 | 0.0168 |
| Positive control: IL-1β 500 pg/ml | 35 ± 3 | 0.0077 |
| Positive control: dibutyryl cAMP 2 mM | 87 ± 8 | 0.0056 |

It will be noted that two series of experiments were performed for ciceritol; one blank control and two positive controls were also effected for these experiments as indicated, in Table II, the ciceritol being that of Example 4.

The three batches of Tephrosia purpurea oligosaccharides, which are the subject of the experiments in Table I, exhibit a dose-dependent stimulation of the release of β-endorphin by normal human keratinocytes in the range 1-25 μg/ml.

The maximum effect is obtained at a concentration of 5 μg/ml.

Furthermore, the experiments performed hitherto on the two molecules present in Tephrosia purpurea extracts, namely stachyose and ciceritol, which are the subject of Table II, have demonstrated a stimulating effect on the production of β-endorphin by these same cells.

In the experiments reported in the context of this Example, the positive controls, IL-1β and dibutyryl CAMP, also stimulated the production of β-endorphin by normal human keratinocytes in culture, but with different amplitudes according to the cellular strains used.

Under these conditions, oligosaccharides, particularly in the form of an extract rich in oligosaccharides—in this case a Tephrosia extract—or the isolated substances stachyose and ciceritol, significantly induce the synthesis of β-endorphin and can thus be used for the manufacture of cosmetic products for the care of sensitive skin, these products being intended for combating skin sensitivity and uncomfortable reactions, providing a sensation of well-being or having a soothing, anti-irritant, local analgesic or antipruritic effect.

Within the context of a pharmaceutical application for the treatment of a pathological condition, oligosaccharides, particularly in the form of a plant extract and in particular a Tephrosia extract, or stachyose or ciceritol, will be useful for the manufacture of pharmaceutical products, notably dermatological products.

Various Examples of cosmetic and pharmaceutical compositions, notably dermatological compositions, are given below. All the components are indicated in percentages by weight, unless indicated otherwise.

### EXAMPLE 6

### Soothing After-Sun Lotion

This soothing lotion is obtained from the following components in the conventional manner well known to those skilled in the art:

| | |
|---|---|
| Tephrosia purpurea extract I₁ of Example 1 | 0.2 |
| ceramide II | 0.5 |
| glycerol | 4 |
| tocopherol acetate | 0.2 |
| liquorice extract | 0.2 |
| excipient | qsp 100 |

This soothing lotion, used after sunbathing, soothes the skin.

### EXAMPLE 7

### Body Firming and Relaxing Gel

This gel is prepared from the following components:

| | |
|---|---|
| Tephrosia purpurea extract I₁ of Example 1 | 1 |
| madecassoside | 0.2 |
| Sapindus mukurossi extract | 0.2 |
| wheat proteins | 2 |
| glycerol | 2 |
| gelling excipient | qsp 100 |

This body firming gel imparts sensations of well-being and pleasure during application by massage.

### EXAMPLE 8

### Night Emulsion with Tightening and Relaxing Effects

This emulsion is prepared from the following components:

| | |
|---|---|
| ciceritol of Example 4 | 0.1 |
| madecassoside | 0.1 |
| sapindosides | 0.1 |
| grape seed OPC | 0.5 |
| emulsified excipient | qsp 100 |

This fine emulsion with tightening effects is on the oval of the face, relaxes the lines, improves the well-being of the skin and makes the face look younger in the morning.

### EXAMPLE 9

### Toning Massage Cream for Sensitive and Irritable Skin

| | |
|---|---|
| Tephrosia purpurea extract I₁ of Example 1 | 1 |
| ginseng extract | 0.1 |
| ergothioneine | 0.2 |
| emulsified greasy excipient for massage | qsp 100 |

This cream for sensitive skin tones the epidermis and relaxes the body.

### EXAMPLE 10

### Fine Emulsion for Regenerating and Relaxing Sensitive and Delicate Skin

| | |
|---|---|
| stachyose | 0.35 |
| retinol palmitate | 0.1 |
| tocopherol acetate | 0.2 |
| soya sapogenols | 0.1 |
| madecassoside | 0.1 |
| sun filters | 8 |
| excipient | qsp 100 |

This fine regenerating emulsion stimulates the epidermal metabolism and restores radiance and youthfulness.

### EXAMPLE 11

### Analgesic and Antipruritic Dermatological Emulsion-Cream

| | |
|---|---|
| ciceritol | 0.2 |
| stachyose | 0.2 |
| emulsified excipient | qsq 100 |

This cream, applied to the areas of skin in question, soothes aches and eliminates or reduces itching of diverse origins.

1. Use of at least one oligosaccharide comprising from 2 to 6 sugars and containing at least 2 galactose units, preferably 2 vicinal galactose units and more preferably two vicinal galactose units at the end of the chain, or of a plant extract containing it, as a cosmetic or dermatological agent for stimulating the production of beta-endorphin in the skin.

2. Use according to claim 1, characterized in that the above-mentioned oligosaccharide is stachyose.

3. Use according to claim 1, characterized in that the above-mentioned oligosaccharide is ciceritol.

4. Use according to one of claims 1 to 3, characterized in that the above-mentioned oligosaccharide is contained in a plant extract, the plant preferably being selected from the group consisting of Tephrosia, soya, chick pea, lupin and lentil.

5. Use according to claim 4, characterized in that the oligosaccharide is contained in an extract of seeds of the species Tephrosia purpurea.

6. Use according to one of the preceding claims, characterized in that the oligosaccharide or the plant extract, expressed by dry weight in this case, is present at a concentration of between 0.0001% and 10% by weight of a cosmetic or dermatological composition, based on the total weight of said composition.

7. Use according to one of the preceding claims, characterized in that the oligosaccharide or the plant extract containing it, expressed by dry weight in this case, is present at a concentration of between 0.01% and 5% by weight of a cosmetic or dermatological composition containing it, based on the total weight of said composition.

8. Use according to one of the preceding claims, characterized in that the above-mentioned oligosaccharide, or a plant extract containing it, is combined with another cosmetically or dermatologically acceptable active substance preferably selected from the group consisting of vitamin A and its esters, particularly vitamin A palmitate; an alpha-hydroxy acid, particularly salicylic acid and its derivatives or lactic, glycolic or malic acid; an inhibitor of the enzyme PLA2, such as an extract of the plant Phellodendron amurense or the plant Azadirachta indica; a substance with anti-inflammatory activity, such as 18B-glycyrrhetinic acid, an extract of the plant Glycyrrhiza glabra; a substance with immunomodulating activity, such as a glycan; a surfactant, particularly of the laurylsulfate family; an alkaloid substance, preferably a bisbenzylisoquinoline and particularly oxyacanthine or cepharanthine; a PAF inhibitor, particularly a Gingko bilobe extract; and an inhibitor of PGE2 enzymes.

9. Use according to any one of the preceding claims, characterized in that the above-mentioned oligosaccharide, or a plant extract containing it, is applied to the skin in order to care for sensitive skin, notably to reduce or eliminate uncomfortable reactions, provide a sensation of well-being or exert a local analgesic action.

10. Method of cosmetic care, characterized in that it comprises the application, to the areas of skin in question, of a cosmetically effective amount of at least one oligosaccharide comprising from 2 to 6 sugars and containing at least 2 galactose units, preferably 2 vicinal galactose units and more preferably two vicinal galactose units at the end of the chain, or of a plant extract containing it, optionally in a cosmetically acceptable excipient, the oligosaccharide or the plant extract containing it preferably being as defined in any one of claims 2 to 9.
